(19) United States
(12) Patent Application Publication
(10) Pub. No.: US 2026/0207202 A1
(43) Pub. Date: Jul. 23, 2026

(54) EMBOLIC PROTECTION SYSTEM

(71) Applicant: Terumo Corporation, Tokyo (JP)

(72) Inventors: Cang Lam, Irvine, CA (US); James Shimabukuro, Aliso Viejo, CA (US); Yoshio Kawashima, Tokyo (JP); Frank Louro, Mission Viejo, CA (US); Tahj Spigner, Costa Mesa, CA (US)

(73) Assignee: Terumo Corporation, Tokyo (JP)

(21) Appl. No.: 19/139,830

(22) PCT Filed: Dec. 21, 2023
(86) PCT No.: PCT/US2023/085465

§ 371 Date: Jun. 16, 2025

Related U.S. Application Data
(60) Provisional application No. 63/476,533, filed on Dec. 21, 2022.

(51) Int. Cl. A61B 17/12

(52) U.S. Cl. CPC A61B 17/12177

(57) ABSTRACT

An embolic protection system for delivering, deploying, and retrieving a filter configured to capture particles which may be dislodged during a medical procedure. The embolic protection system may include an embolic protection device for capturing the particles, a delivery catheter assembly for delivering the embolic protection device to a target location within a vasculature, and a retrieval catheter assembly for retrieving the embolic protection device along with any captured particles from the target location after use. The embolic protection device may include a filter which is movably connected to a guidewire such that the filter may move both axially and rotationally with respect to the guidewire after deployment and thereby reduce the risk of vasospasm or vessel dissection.

### RELATED APPLICATIONS

This application claims priority to U.S. Provisional Application Ser. No. 63/476,533 filed Dec. 21, 2022, entitled Embolic Protection System, which is hereby incorporated herein by reference in its entirety.

### BACKGROUND

During certain medical procedures, such as but not limited to carotid artery stent procedures, a physician's surgical tools can sometimes dislodge embolic particles. Such embolic particles may typically include thrombus, atheroma, and lipids which, once dislodged, can cause blockages in downstream vessels. Hence, these embolic particles can result in serious complications, such as stroke or even death.

One method for reducing the risk of such complications is to deploy an embolic protection device such as a filter downstream of a surgical treatment site, thereby catching any particles that may become dislodged. Once caught, the filter may be closed and withdrawn from the patient, such that the captured embolic particles do not escape the filter.

It may be desirable that such filters may be adjustable in one or more manners to reduce the risk of various undesirable conditions, such as vasospasm or vessel dissection. It may also be desirable that such filters may include an integrated guidewire such that a separate guidewire is not necessary during the procedure, may comprise radiopaque wires to improve visibility, and may include an anti-clot surface treatment to reduce clot formation.

### SUMMARY

Disclosed herein is an embolic protection system which may be delivered to a target location within a patient, deployed to capture any dislodged particles, and retrieved from the patient.

In an example embodiment, the embolic protection system may comprise an embolic protection device for capturing any dislodged particles, a delivery catheter assembly for delivering the embolic protection device, and a retrieval catheter assembly for retrieving the embolic protection device including any captured particles.

In an example embodiment, the embolic protection device may comprise a filter including a braided mesh and/or one or more structural wires.

In an example embodiment, the embolic protection device may comprise one or more clamps for connecting the embolic protection device to an elongated member such as a guidewire.

In an example embodiment, one or more of the clamp(s) may be movably connected to the guidewire.

In an example embodiment, a tubular member such as an elongated cylindrical member may extend distally from a distal clamp of the embolic protection device for improved visualization.

In an example embodiment, the tubular member may comprise a variable durometer along its length.

In an example embodiment, a first portion of the tubular member may comprise a first durometer and a second portion of the tubular member may comprise a second durometer.

In an example embodiment, a proximal portion of the tubular member may comprise a durometer that is greater than that of a distal portion of the tubular member.

In an example embodiment, a proximal portion of the tubular member may comprise a first material and a distal portion of the tubular member may comprise a second material.

In an example embodiment, the first material may comprise PEBAX 53D and the second material may comprise PEBAX 35D.

In an example embodiment, the filter may be adjustable between a radially compressed configuration and a radially expanded configuration.

In an example embodiment, the filter may comprise a conical shape when in the radially expanded configuration.

In an example embodiment, the filter may be connected to one or more clamps such that the filter may move with respect to an underlying elongated member when the filter is in the radially expanded configuration.

In an example embodiment, the filter may be connected to the elongated member by a pair of clamps including a first clamp connected to a proximal end of the filter and a second clamp connected to a distal end of the filter.

In an example embodiment, the filter may move axially with respect to the elongated member when the filter is in the radially expanded configuration.

In an example embodiment, the filter may move rotationally with respect to the elongated member when the filter is in the radially expanded configuration.

In an example embodiment, the filter may move both rotationally and axially with respect to the elongated member when the filter is in the radially expanded configuration.

In an example embodiment, the filter may comprise a plurality of wire pairs.

In an example embodiment, the filter may comprise a braided mesh and one or more structural wires. The one or more structural wires may comprise one or more drawn filled tubing (DFT) wires. The structural wires may comprise twinned pairs of wires such that a one or more wire pairs form at least a portion of the filter.

In an example embodiment, a stopper may be connected to the elongated member for limiting movement of the filter with respect to the elongated member. The stopper may function to limit axial translation of the filter. The stopper may also or alternatively function to interconnect sections of the elongated member.

In an example embodiment, the filter (e.g., the structural wire(s) and/or braided mesh) may be treated with an anti-clot surface treatment to aid in prevention of clot formation during use.

In an example embodiment, a radiopaque band, wire, or coil may be positioned around a distal end or portion of the elongated member to aid in visualizing the distal end of the elongated member during use.

In an example embodiment, the one or more structural wires may extend across a length of the filter and the meshed braid may extend along only about 40%-60% of the length of the filter.

In an example embodiment, an embolic protection system may comprise a filter assembly comprising a guidewire and a filter movably connected the guidewire and a delivery catheter assembly comprising a housing for storing the filter assembly prior to deployment and a release wire connected to the housing for retracting the housing from around the filter.

In an example embodiment, a deployment handle may be connected to the release wire so as to allow the release wire to be pushed or pulled by one-handed operation.

In an example embodiment, the deployment handle may comprise a trigger movably or slidably connected within a slot such that the trigger may be retracted proximally to retract the release wire and thereby deploy the filter of the embolic protection device.

In an example embodiment, the deployment handle may include a locking mechanism for locking the trigger in the undeployed configuration and thereby prevent premature deployment of the embolic protection device.

In an example embodiment, the housing may include at least one marker band. The at least one marker band may be positioned at or near a distal end of the housing.

In an example embodiment, the embolic protection system may further comprise a retrieval catheter assembly for retrieving the embolic protection device along with any captured particles contained therein after use.

In an example embodiment, the retrieval catheter assembly may include one or more marker bands to visualize when the filter is fully contained within the retrieval catheter assembly.

In an example embodiment, a distal mouth of the retrieval catheter may comprise an inner sloped surface or inwardly tapered distal end so as to prevent fraying of the filter as the filter enters the distal mouth of the retrieval catheter.

In an example embodiment, an adjustment handle may be connected to the retrieval catheter so as to selectively deflect or movably adjust a distal end of the retrieval catheter and thereby improve navigability when positioning the retrieval catheter to retrieve the filter.

In an example embodiment, the retrieval catheter may be flushed with a fluid (e.g., saline) while in its original packaging by filling a syringe with the fluid, fluidly connecting the syringe to the retrieval catheter (e.g., through use of flexible tubing), and dispelling the fluid from the syringe such that the fluid flushes out the retrieval catheter.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure. These and other aspects, features and advantages of which embodiments of the disclosure are capable of will be apparent and elucidated from the following description of embodiments of the present disclosure, reference being made to the accompanying drawings, in which:

FIG. 1 illustrates the embolic protection device in a radially expanded configuration, in accordance with an example embodiment of the present disclosure.

FIG. 2 illustrates a side view of an embolic protection system in a radially compressed configuration, in accordance with an example embodiment of the present disclosure.

FIG. 3 illustrates a side view of an embolic protection device of an embolic protection system in a radially expanded configuration including a filter which is movably connected to an elongated member such as a guidewire, in accordance with an example embodiment of the present disclosure.

FIG. 4 illustrates a first perspective view of a filter for use with an embolic protection device in an expanded configuration, in accordance with an example embodiment of the present disclosure.

FIG. 5 illustrates a second perspective view of a filter for use with an embolic protection device in an expanded configuration, in accordance with an example embodiment of the present disclosure.

FIG. 6 illustrates a side view of a delivery catheter of an embolic protection system including interconnected pull and push assemblies, in accordance with an example embodiment of the present disclosure.

FIG. 7 illustrates a side view of a pull assembly of a delivery catheter of an embolic protection system, in accordance with an example embodiment of the present disclosure.

FIG. 8 illustrates a side view of a push assembly of a delivery catheter of an embolic protection system, in accordance with an example embodiment of the present disclosure.

FIG. 9 illustrates a side view of a retrieval catheter of an embolic protection system which may be utilized to retrieve the embolic protection device, in accordance with an example embodiment of the present disclosure.

FIG. 10 illustrates a close-up view of a frame and braiding of an embolic protection device, in accordance with an example embodiment of the present disclosure.

FIG. 11 illustrates a side view of another example of an embolic protection device of an embolic protection system, in accordance with an example embodiment of the present disclosure.

FIG. 12A illustrates an upper perspective view of a deployment handle of an embolic protection system in an undeployed configuration, in accordance with an example embodiment of the present disclosure.

FIG. 12B illustrates an upper perspective view of a deployment handle of an embolic protection system in a deployed configuration, in accordance with an example embodiment of the present disclosure.

FIG. 12C illustrates a side view of a deployment handle of an embolic protection system in an unlocked configuration, in accordance with an example embodiment of the present disclosure.

FIG. 12D illustrates a top view of a deployment handle of an embolic protection system in a locked configuration, in accordance with an example embodiment of the present disclosure.

FIG. 13A illustrates a perspective view of a retrieval catheter positioned to retrieve an embolic protection device of an embolic protection system, in accordance with an example embodiment of the present disclosure.

FIG. 13B illustrates a perspective view of an embolic protection device being retrieved by a retrieval catheter of an embolic protection system, in accordance with an example embodiment of the present disclosure.

FIG. 14 illustrates a side view of an adjustment handle for use with the retriever catheter of an embolic protection system, in accordance with an example embodiment of the present disclosure.

FIG. 15 illustrates a side view of a flushing system for flushing the retriever catheter of an embolic protection system, in accordance with an example embodiment of the present disclosure.

FIG. 16A illustrates a proximal region of the delivery catheter having multiple lumens, in accordance with an example embodiment of the present disclosure.

FIG. 16B illustrates a cross section B-B of the delivery catheter of FIG. 16A, in accordance with an example embodiment of the present disclosure.

FIG. 16C illustrates a cross section C-C of the delivery catheter of FIG. 16A, in accordance with an example embodiment of the present disclosure.

FIG. 17 illustrates an example of a distal end of the guidewire of the embolic protection device, in accordance with an example embodiment of the present disclosure.

### DETAILED DESCRIPTION

Specific embodiments of the disclosure will now be described with reference to the accompanying drawings. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. The terminology used in the detailed description of the embodiments illustrated in the accompanying drawings is not intended to be limiting of the disclosure. In the drawings, like numbers refer to like elements.

For the purposes of the terminology described below, the terms clot, thrombus, embolus, and obstruction can be used synonymously.

For the purposes of this specification, use of the terms “about”, “around”, or “approximately” when referring to a value may be understood to mean within 5% of the stated value (either greater or lesser), inclusive.

Disclosed herein are example embodiments of an embolic protection system which may include, e.g., an embolic protection device for capturing dislodged embolic particles, a delivery catheter assembly for delivering the embolic protection device, and a retrieval catheter assembly for retrieving the embolic protection device including any captured particles.

The embolic protection device may be deployed in a vasculature, such as the carotid artery, distally to a location where a medical procedure known to have a potential for dislodging one or more particles, such as a stenting or angioplasty procedure, is being performed. If one or more particles are dislodged during the procedure, the embolic protection device may capture such dislodged particles so that they may be removed safely from the body.

The embolic protection device may comprise an elongated member, such as an integrated guidewire, to which a filter may be movably connected. The elongated member may extend completely through the filter and extend both distally and proximally therefrom. The elongated member may comprise two or more distinct sections which are interconnected together. For example, the elongated member may comprise a proximal guidewire and a distal guidewire.

A distal section of the elongated member may include a radiopaque marker to ease tracking during navigation to a target location within the body. The radiopaque marker may comprise a radiopaque material which is known to be visible by various imaging devices. The radiopaque marker may comprise a band or a coil which is affixed to a distal portion of the elongated member, such as to a distal end thereof, by various methods known in the art such as welding.

The filter may comprise a frame. The frame may include one or more structural wires which are weaved, braided, or coiled to form a substantially conical structure having an internal cavity. At least a portion of the one or more structural wires may be connected to a braid, such as a meshed braid, for capturing any dislodged particles. For example, a distal half of the filter may include the braid for capturing debris, and a proximal half of the filter may not include the braid, but may instead include only one or more structural wires, so as to allow dislodged particles to enter the internal cavity of the filter and be captured therein.

The filter may be movably connected to the elongated member. For example, the filter may be axially movable along the elongated member and/or rotationally movable with respect to the elongated member. Such movement of the frame with respect to the elongated member may reduce potential for vasospasm or dissection while manipulating the elongated member, such as during positioning of the filter.

The filter may be connected to the elongated member by one or more clamps. For example, the filter may be connected to the elongated member at the filter's proximal end by a first clamp and at the filter's distal end by a second clamp. The filter may be fixedly attached to the clamps. One or more of the clamps may be movably connected to the elongated member such that the clamps may move axially slide and/or rotate with respect to the elongated member and thereby allow the same movement(s) by the filter.

The filter may be adjustable between at least two configurations. For example, the filter may be adjustable between a radially compressed configuration and a radially expanded configuration. In the radially compressed configuration, the filter may be compressed to fit within a tubular housing such as within a delivery catheter. In the radially expanded configuration, the filter may be expanded to form a substantially conical shape having a partially-exposed internal cavity for capturing any dislodged particles.

A distal tubing tip may be connected to the clamp positioned at or near the distal end of the filter. The distal tubing tip may be positioned at least partially over or around the elongated member. The distal tubing tip may be composed of a radiopaque material to provide visualization of the distal end of the filter by various imaging devices and thereby aid with tracking.

A stopper may be connected to the elongated member distally with respect to the clamp positioned at or near the proximal end of the filter. The stopper may function to limit axial translation of the filter. The stopper may also provide the function of connecting different sections of the elongated member, such as connecting a distal guidewire with a proximal guidewire which, together, may form the elongated member.

The delivery catheter assembly may be utilized to transport and deploy the embolic protection device at a target location during a medical procedure. The delivery catheter assembly may be tracked to a target location, such as a location in the carotid artery, with the embolic protection device positioned therein. A release wire may then be pulled which causes a housing of the delivery catheter assembly to retract and thereby allow the embolic protection device to expand within a target vessel.

To achieve the release wire deployment, the delivery catheter assembly may comprise a pair of subassemblies that can move in the axial direction independently of one another. For example, the delivery catheter assembly may comprise a pull subassembly and a push subassembly, with the pull and push subassemblies being interconnected with each other to form the unitary delivery catheter assembly.

The pull subassembly may comprise a housing, a marker band, a distal shaft, and a release wire. The housing may comprise a tubular member in which the embolic protection device is positioned prior to deployment. The marker band may be composed a radiopaque material to indicate when the embolic protection device is fully within the housing. The distal shaft may comprise a cylindrical member (solid or tubular) which connected the housing to the release wire. The release wire may comprise an elongated wire which may retract the housing when pulled.

The push subassembly may comprise a guidewire lumen, a cover tube, and a hypotube. The guidewire lumen may serve as a hard stop for the embolic protection device when in the housing. The guidewire lumen may also function to push the filter out of the housing as the housing retracts. The cover tube may cover the main interface between the push and pull assembly. The hypotube may store the release wire.

The retrieval catheter assembly may be used to retrieve the embolic protection device after use, along with any captured particles. The retrieval catheter assembly may be advanced to the embolic protection device and the embolic protection device may be pulled into the retrieval catheter assembly. Both the retrieval catheter assembly and the embolic protection device may then be removed from the body together.

The retrieval catheter assembly may comprise a distal mouth, a housing and cover tube, one or more marker bands, and a proximal shaft. The distal mouth may comprise a circular opening having a larger inner diameter than the housing to minimize or avoid fraying of the braid of the filter during retrieval. The housing and cover tube may comprise a tubular member within which the collapsed embolic protection device may be stored during retrieval. The one or more marker bands may be connected to the housing and cover tube at different locations to aid in visualizing when the embolic protection device is fully within the housing. The proximal shaft may function to connect the housing to a structural wire that forms the remainder of the length of the retrieval catheter assembly.

Specific example embodiments are described further below. However, it should be understood that any of the features from any of the embodiments can be mixed and matched with each other in any combination. Hence, the present disclosure should not be restricted to only these embodiments, but any broader combination thereof.

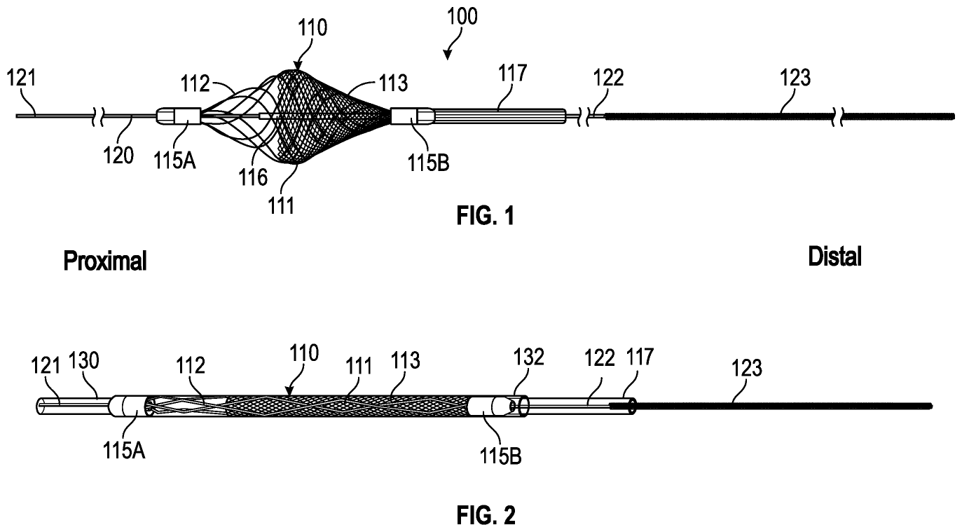

FIGS. 1-2 illustrate an example embodiment of an embolic protection system 100 including an embolic protection device 110.

FIG. 1 illustrates the embolic protection device 110 in a radially expanded configuration. In the radially expanded configuration, a filter 111 may radially expand into an expanded shape. In some embodiments, the radially expanded configuration may be utilized when the filter 111 is deployed for use in capturing dislodged particles. In the example embodiment shown in the figures, the expanded shape may comprise a substantially conical shape. However, it should be appreciated that other shapes may be utilized. It should further be appreciated that, depending upon axial and/or rotational movement of the filter 111 as discussed below, the overall shape of the filter 111 may vary during use to suit different situations, vessels, positionings, and the like.

FIG. 2 illustrates a side view the embolic protection device 110 in a radially compressed configuration. In some embodiments, in the radially compressed configuration, the filter 111 may be radially compressed into a substantially linear or tubular shape such as shown in the figure. In such a radially compressed configuration, the filter 111 may be sized and shaped to fit within a tubular housing 132 of a catheter. The radially compressed configuration may be utilized when the filter 111 is to be delivered to and/or retrieved from a target location within a patient's body. For example, the radially compressed configuration may be utilized to fit the filter 111 within a delivery catheter 130 for delivery to a target location and, after use, to fit the filter 111 within a retrieval catheter 140 (not shown) for retrieval from a target location, along with any captured dislodged particles.

Continuing to reference FIGS. 1-2, in some embodiments, the embolic protection device 110 may include an elongated member such as a guidewire 120. Although the term “guidewire” is utilized herein with relation to the elongated member, it should be appreciated that various other types of elongated members may be utilized in connection with the embolic protection device 110.

In some embodiments, the guidewire 120 may be integrated with the embolic protection device 110 so as to ease use of the embolic protection system 100 during a medical procedure. Previously, embolic shields and the like have required a separate guidewire to be advanced to a target location within a patient's body. By instead incorporating an integrated guidewire 120 such as shown in FIG. 1, the example embodiment may negate the need for such an additional device and thus increase efficiency of use of the embolic protection system 100.

As best shown in FIGS. 1-2, the guidewire 120 may comprise a proximal section 121 which extends outwardly in a proximal direction from the filter 111 and a distal section 122 which extends outwardly in a distal direction from the filter 111. In some example embodiments as discussed below, the proximal and distal sections 121, 122 may comprise separate elongated members which may be interconnected together to form a single, unitary guidewire 120. The length of the respective proximal and distal sections 121, 122 with respect to the total length of the guidewire 120 may vary in different embodiments, and thus should not be construed as limited by the example embodiments shown in the figures.

In some embodiments, to aid in visualization, the distal section 122 may include a marker 123 composed of a radiopaque material. The marker 123 may comprise various configurations including, for example, a wire coiled around at least a portion of the distal section 122. The type of radiopaque material forming such a marker 123 may vary and may include, e.g., platinum. In this manner, the distal end 122 of the guidewire 120 may be visualized by various imaging devices known in the art to thereby aid in tracking the guidewire 120 as it is being navigated to a target location within a patient's body. In some embodiments, the proximal section 121 may alternatively or additionally include a marker 123 composed of a radiopaque material.

In the example embodiment illustrated in FIGS. 1-2, it can be seen that the marker 123 may comprise a wire which is coiled around the proximal section 121 of the guidewire 120 to form a coil marker. However, various other configurations may be utilized in different embodiments. By way of example, the marker 123 may instead comprise a marker band which is positioned radially around the proximal section 121 and/or the distal section 122 of the guidewire 120. As yet another example, the proximal section 121 and/or the distal section 122 of the guidewire 120 itself may at least partially be formed from a radiopaque material.

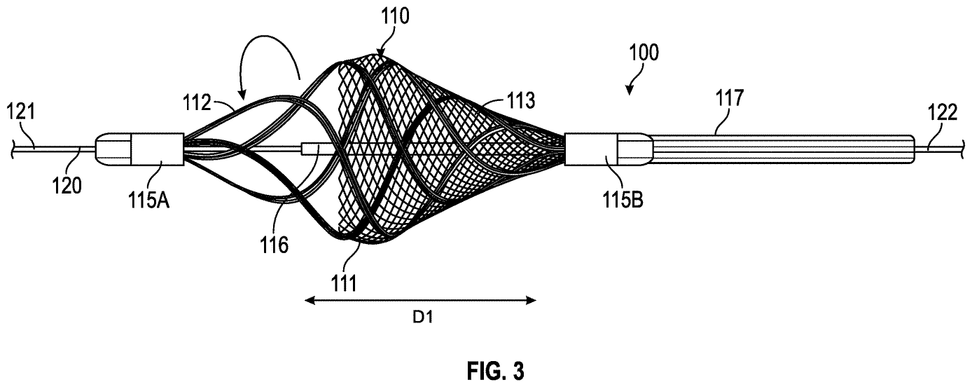

FIG. 3 is a sideview of an example embodiment of an embolic protection device 110 in a radially expanded configuration including a filter 111 which is movably connected to an elongated member such as a guidewire 120. As illustrated by the directional arrows D1, the filter 111 may be adjustably movable along a longitudinal axis such that the filter 111 may be axially moved with respect to the underlying guidewire 120. Alternatively or additionally, the filter 1111 may be adjustably movable to rotate about the longitudinal axis such that the filter 111 may be rotated with respect to the underlying guidewire 120. Although FIG. 3 illustrates a filter 111 exhibiting both axial and rotational movement, it should be appreciated that, in some embodiments, the filter 111 may only be movable axially or rotationally, rather than both.

The manner by which the filter 111 is movably connected to the guidewire 120 may vary in different embodiments. In the example embodiment shown in FIG. 3, it can be seen that the filter 111 is attached directly to a pair of clamps 115A, 115B, with one or both of the clamps 115A, 115B being movably (rotationally and/or axially) connected to the guidewire 120. In some embodiments, the clamps 115A, 115B may comprise various types of connectors and thus should not be construed as limited in scope to the cylindrical, capped tubular members as shown in the example embodiments of the drawings.

In an example embodiment as shown in FIGS. 3-5, a first clamp 115A may be connected to a proximal end of the filter 111 and a second clamp 115B may be connected to a distal end of the filter 111. In some embodiments, the clamps 115A, 115B may each include an internal opening through which the guidewire 120 extends. In some embodiments, each of the clamps 115A, 115B may be free to axially move along a longitudinal axis towards or away from each other. Additionally, or alternatively, in some embodiments, each of the clamps 115A, 115B may be free to rotate about the guidewire 120.

As best shown in FIG. 3, in some embodiments, a filter stopper 116 may be connected to the guidewire 120 between the proximal and distal clamps 115A, 115B. In some embodiments, the filter stopper 116 may comprise a tubular member which is fixed to the guidewire 120 between the clamps 115A, 115B. The filter stopper 116 may be utilized to set a minimum distance between the clamps 115A, 115B such that the filter's 111 axial movement along the guidewire 120 is limited within a set range. Thus, the filter stopper 116 may function to prevent the clamps 115A, 115B from converging together and thus set a minimum length along which the filter 111 may be compressed axially.

The example embodiment of FIG. 3 illustrates that the length of the filter stopper 116 may be greater than half a length of the filter 111. It should be appreciated, however, that the length of the filter stopper 116 may vary in different embodiments and thus should not be construed as limited by the example embodiments shown in the figures. For example, the filter stopper 116 may be longer or shorter than shown in the figures. Additionally, although the figures illustrate that the filter stopper 116 may be positioned primarily under the meshed portion of the filter 111, alternate configurations may be utilized in different embodiments.

Continuing with reference to FIG. 3, in some embodiments, a tubular member 117 may be positioned over at least a portion of the guidewire 120 so as to increase stiffness and to create a transition from a higher stiffness segment of the filter 111 to a lower stiffness segment of the guidewire 120. The tubular member 117 may at least partially cover, be adjacent to, and/or be attached to the distal clamp 115B such that the tubular member 117 covers at least a portion of the distal section 122 of the guidewire 121 such as shown in FIG. 3. However, in some embodiments, the tubular member 117 may alternatively or additionally be positioned to cover or be adjacent with the proximal clamp 115A.

FIG. 3 illustrates an example embodiment in which the tubular member 117 is positioned around the guidewire 120 and adjacent to the distal clamp 115B though, in some examples as stated above, a tubular member 117 may additionally or alternatively be positioned around the guidewire 120 and adjacent to the proximal clamp 115A. In some embodiments, the tubular member 117 may be attached directly to the proximal clamp 115A and/or the distal clamp 115B. In other embodiments, the tubular member 117 may be attached directly to the guidewire 120 adjacent to the proximal clamp 115 and/or the distal clamp 115B. The tubular member 117 may be composed of various materials, including but not limited to polymeric materials or thermoplastic elastomers such as polyether block amide (PEBAX). The length of the tubular member 117 may vary in different embodiments and thus should not be construed as limited by the example embodiments shown in the figures.

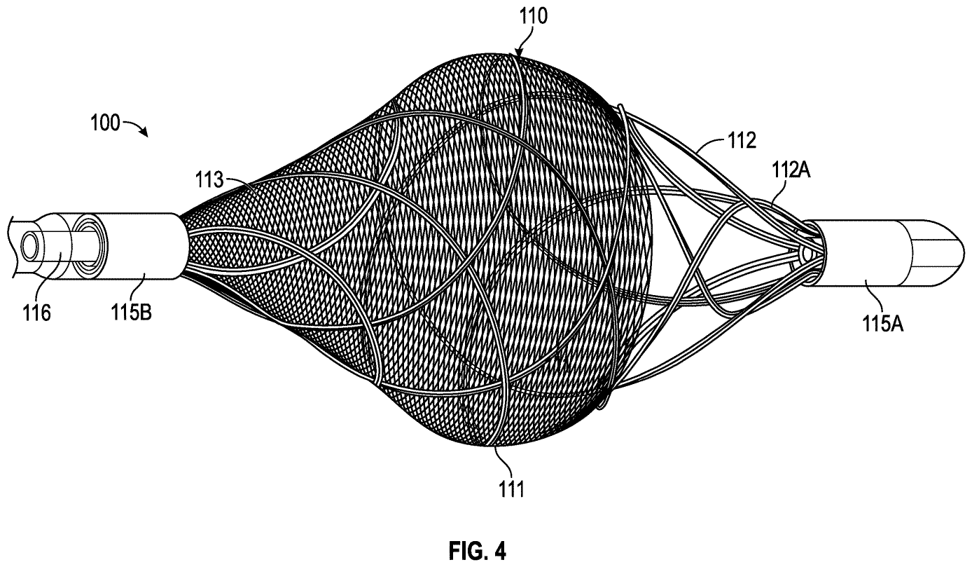

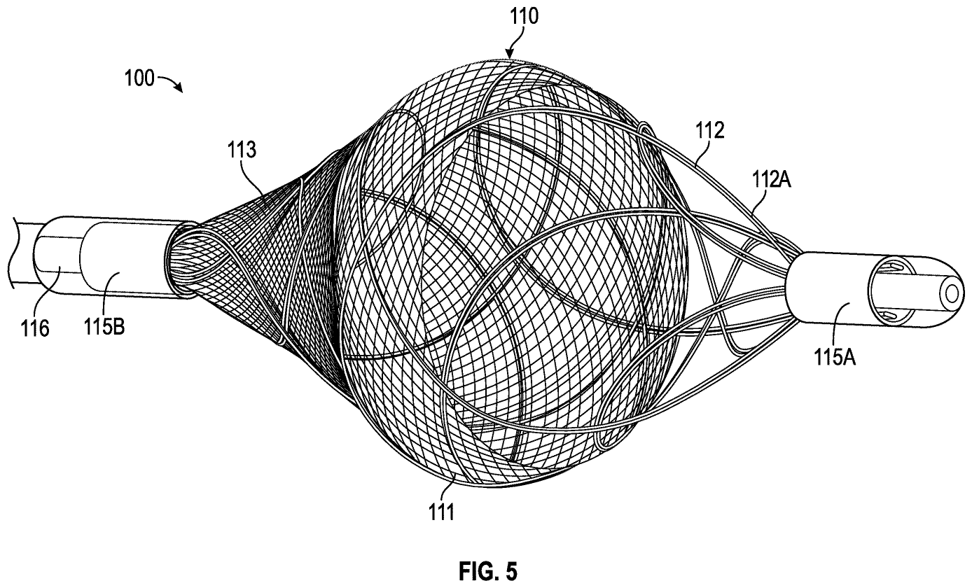

FIGS. 4-5 illustrate an example embodiment of a filter 111 for use with an embolic protection device 110. FIG. 4 is a first perspective view of a filter 111 for use with an embolic protection device 110 in an expanded configuration. FIG. 5 is a second perspective view of a filter for use with an embolic protection device in an expanded configuration.

The filter 111 is illustrated in FIGS. 4-5 in its radially expanded configuration. It should be appreciated that the overall conical shape of the filter 111 illustrated in the figures may vary in different embodiments and thus should not be construed as limiting in scope. For example, the shape of the filter 111 may vary depending upon any axial and/or rotational movement of the filter 111 with respect to the guidewire 120 (not shown), with the effective width or diameter of the filter 111 being made larger or smaller depending on the distance between the clamps 115A, 115B.

As shown in FIGS. 4-5, in some embodiments, the filter 111 may comprise a frame 112. The frame 112 may be formed from one or more structural wires which may be manipulated and heat set into a desired shape, such as the substantially conical shape shown in the figures. Various types of structural wires may be utilized to form the frame.

In an example embodiment, the structural wires forming the frame 112 may be comprised of drawn filled tubing (DFT) wires or other wires formed at least partially from radiopaque material(s). The use of DFT wires for the frame 112 may negate the need for separate radiopaque markers on the frame 112. However, in some embodiments, non-radiopaque structural wires may form the frame 112 and separate radiopaque markers may be attached to various parts of the frame 112.

With reference to FIGS. 4-5, it can be seen that an example embodiment of the filter 111 may comprise a braid 113 which is connected to the frame 112. In some embodiments, the braid 113 may comprise a meshed braid as shown in the figures, or other configurations may be utilized. The braid 113 may function to capture any dislodged particles within the internal cavity of the filter 111 for retrieval.

In the example embodiments shown in the figures, it can be seen that the braid 113 may cover slightly more than half of a length of the filter 111. Such a configuration should not be construed as limiting in scope. In some embodiments, the braid 113 may cover less than half of the length of the filter 111. By way of example, the braid 113 may cover 10%, 20%, 30%, 40%, 50%, 60%, 70%, 80%, or 90% of the filter 111 in different embodiments.

In the example embodiment shown in the figures, it can be seen that around 40% of the filter 111 is not covered by the braid 113. In this manner, the frame 112 may be left exposed so as to allow any dislodged particles to enter the filter 111 and be captured within the braid 113. In an example embodiment, the braid 113 may be positioned to cover a distal portion of the frame 112 of the filter 111. However, in some embodiments, the reverse configuration may be utilized.

In an example embodiment, the frame 112 may be comprised of about 16 structural DFT wires having a diameter of about 0.0030 inches. In some embodiments, the braid 113 may be comprised of about 88 smaller Nitinol wires having a diameter of about 0.0014 inches. However, these values are merely for exemplary purposes and should not be construed as limiting in scope. More or less wires may be utilized to form the frame 112 and/or braid 113. Differently-sized wires may also be utilized to form the frame 112 and/or braid 113. Further, different materials other than DFT and Nitinol may be used to form the frame 112 and braid 113, respectively.

The manner by which the braid 113 is secured to the frame 112 may vary in different embodiments. Various methods known in the art for securing a braid 113 to a frame 112 may be utilized, such as but not limited to welding the like. The frame 112 and/or the braid 113 may also be treated with an anti-clot surface treatment or coating to aid in reducing clot formation when the filter 111 is in use.

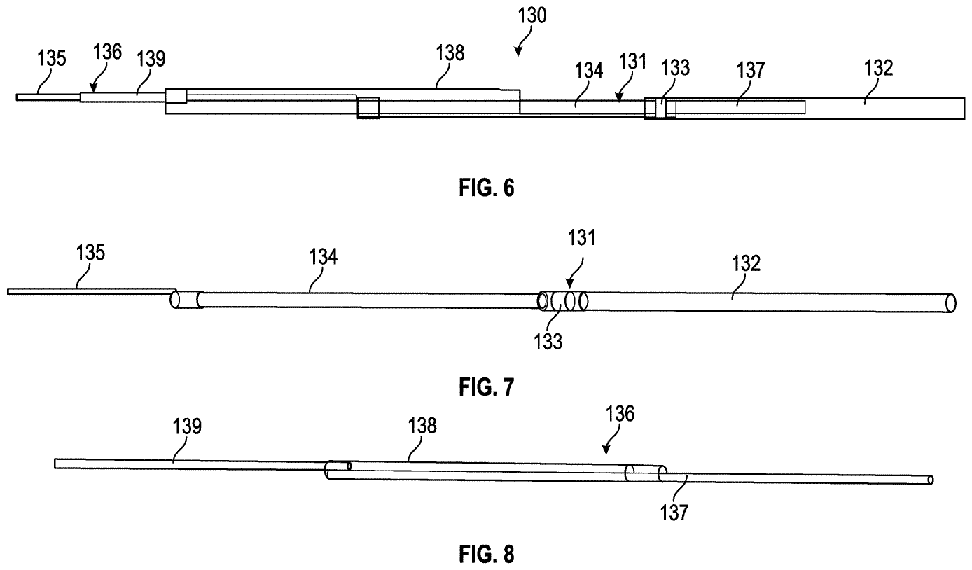

FIGS. 6-8 illustrate an example embodiment of a delivery catheter 130 which may be utilized to deliver the embolic protection device 110, including the filter 111, to a target location within the body, with the proximal side being on the left-hand of the figures and the distal side being on the right-hand of the figures. FIG. 6 illustrates a side view of an example embodiment of the delivery catheter 130 including interconnected pull and push assemblies 131, 136. FIG. 7 illustrates a side view of an example embodiment of the pull assembly 131 of the delivery catheter 130. FIG. 8 illustrates a side view of an example embodiment of the push assembly 136 of the delivery catheter 130.

As best shown in FIGS. 6-7, the delivery catheter 130 may comprise a pull assembly 131 which may be utilized to retract the delivery catheter 130 from around the embolic protection device 110 and thereby expose and deploy the filter 111. In some embodiments, the pull assembly 131 may comprise a housing 132, a distal shaft 134, and a release wire 135.

In some embodiments, the embolic protection device 110, including the filter 111, may be contained within the housing 132 of the delivery catheter 130 in its radially compressed configuration during delivery to a target location within a patient's body. In some embodiments, the housing 132 may comprise a tubular member such as shown in FIG. 7. The length and width of the housing 132 may vary in different embodiments to suit different embodiments of the filter 111. In some embodiments, the housing 132 may include a marker band 133 at or near its proximal end. The marker band 133 may be composed of a radiopaque material which aids in visualizing and indicating when the filter 111 is completely within the housing 132.

In some embodiments, the distal shaft 134 may comprise a solid or tubular elongated member which is connected between the housing 132 and the release wire 135. Generally, in some embodiments, the filter 111 will not enter within the distal shaft 134 but will instead be positioned exclusively within the housing 132 during delivery. In some embodiments, the release wire 135 may be affixed to a proximal end of the distal shaft 134. Pulling on the release wire 135 may function to retract the housing 132 from around the filter 111 and thereby deploy the filter 111.

As best shown in FIGS. 6 and 8, the delivery catheter 130 may comprise a push assembly 136 which may function to aid in delivery of the filter 111 by pushing the filter 111 out of the housing 132 of the pull assembly 131 as the housing 132 is retracted. In some embodiments, the push assembly 136 may comprise a guidewire lumen 137, cover tubing 138, and hypotube 139 as best shown in FIG. 8. Both the pull and push assemblies 131, 136 may move in the axial direction independently of one another.

With reference to FIG. 8, it can be seen that the guidewire lumen 137 may serve the dual functions of serving as a hard stop for the filter 111 within the housing 132 and pushing the filter 111 out of the housing 132 when the housing 132 is retracted for deployment of the filter 111. In some embodiments, the cover tubing 138 may comprise a tubular member which covers the main interface between the interconnected pull and push assemblies 131, 136 such as shown in FIG. 6. In some embodiments, the hypotube 139 may function to contain the release wire 135 and may be bonded within the proximal end of the cover tubing 138. The guidewire lumen 137 may be similarly secured within the cover tubing 138 while extending distally therefrom.

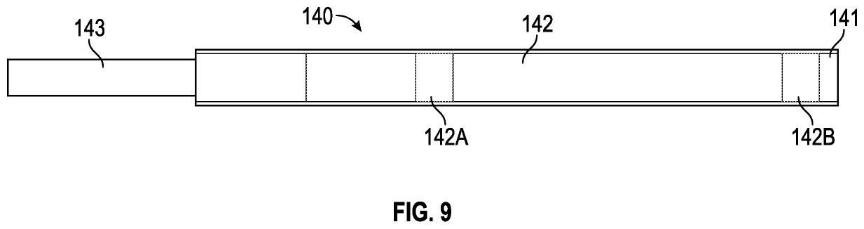

FIG. 9 illustrates a side view of an example embodiment of a retrieval catheter 140 which may be utilized to retrieve the embolic protection device 110, along with any captured particles within the filter 111, from the target location within the body after a medical procedure has been completed. As shown in FIG. 9, an example embodiment of the retrieval catheter 140 may comprise a distal mouth 141, a housing 142, one or more marker bands 142A, 142B, and a proximal shaft 143. The retrieval catheter 140 may comprise a retrieval housing 142 for receiving the filter 111.

In some embodiments, the housing 142 may comprise a tubular member sized to fit the filter 111 therein while in the radially collapsed configuration. In some embodiments, the housing 142 may comprise a distal mouth 141 at its distal end. The distal mouth 141 may have a greater inner diameter than that of the housing 142 so as to minimize fraying of the filter 111 during retrieval.

Referring to FIG. 9, one or more marker bands 142A, 142B composed of a radiopaque material may be connected at least partially around the housing 142 to aid in visualization. For example, the one or more marker bands 142A, 142B may aid in an operator visualizing when the filter 111 is fully within the retrieval housing 142. The number of marker bands 142A, 142B may vary in different embodiments and thus should not be construed as limited in scope by the example embodiments shown in the figures. For example, more or less than the two marker bands 142A, 142B shown in the figures may be utilized.

Continuing to reference FIG. 9, it can be seen that, in an example embodiment, the retrieval housing 142 may include a pair of marker bands 142A, 142B including a first marker band 142A and a second marker band 142B. In some embodiments, the second marker band 142B may be positioned at or near a distal end of the housing 142, near the distal mouth 141. In some embodiments, the first marker band 142A may be axially spaced away from the second marker band 142B towards the proximal end of the retrieval housing 142. The distance between the marker bands 142A, 142B may vary, but generally may be approximately equal to a length of the filter 111 when in its radially compressed configuration.

In some embodiments, the proximal shaft 143 may be connected within a proximal end of the retrieval housing 142 and extend proximally therefrom as shown in FIG. 9. The proximal shaft 143 may function to connect the housing to any structural wire or other elongated member which forms the remainder of the proximal length of the retrieval catheter 140. The respective lengths of the housing 142, proximal shaft 143, and retrieval catheter 140 overall may vary in different embodiments to suit different applications.

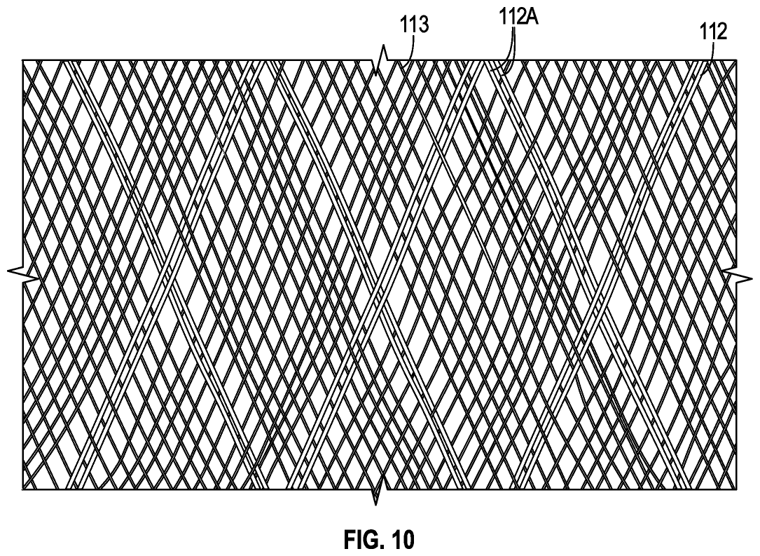

FIG. 10 illustrates a close-up view of the frame 112 and braid 113 portions of an embolic protection device 110. In some example embodiments such as best shown in FIGS. 4-5 and 10, all or a portion of the frame 112 may be formed from one or more twinned pairs of structural wires 112A. Put differently, some or all of the structural wires forming the frame may comprise a pair of wires 112A which are converged or fused together to increase structural integrity. Such twinned pairs of structural wires 112A may provide a supporting structure for the filter 111 in an expanded or collapsed configuration.

The twinned pair of structural wires 112A may be adjustable between at least a collapsed configuration and an expanded configuration. In the collapsed configuration, the twinned pair of structural wires 112A may collapse or compress into a substantially cylindrical shape so as to fit within a delivery device. In the expanded configuration, the twinned pair of structural wires 112A may expand into various shapes, including but not limited to the shape shown in FIGS. 4-5 and 10 in which the outer profile of the expanded twinned pairs of structural wires 112A may have a central portion with a greater diameter which tapers to reduce in diameter towards its proximal and distal portions, resulting in a central bulge.

The twinned pairs of structural wires 112A may extend between the proximal clamp 115A and the distal clamp 115B, with the twinned pair of structural wires 112A at the proximal end of the frame 112 being cinched by or otherwise secured to the proximal clamp 115A and the twinned pair of structural wires 112A at the distal end of the frame 112 being cinched or otherwise secured to the distal clamp 115B.

The filter 111 may be secured to and/or by the twinned pair of structural wires 112A. The twinned pair of structural wires 112A may be positioned along an exterior surface of the filter 111 as shown in the figures or, in some embodiments, may also extend through or across interior regions of the filter 111. The twinned pair of structural wires 112A may be in contact with one or more regions of the exterior surface of the filter 111. The frame 112 may expand and/or collapse in accordance with the filter 111. The expansion and/or collapse of the filter 111 may function to also expand and/or collapse the frame 112 and/or the expansion and/or collapse of the frame 112 may function to also expand and/or collapse the filter 111.

Although not shown, in some embodiments, three or more wires may be paired together in a similar manner to form the structural wires of the frame 112. The use of such a configuration for the structural wires may aid in visibility and provide structural integrity to the filter 111.

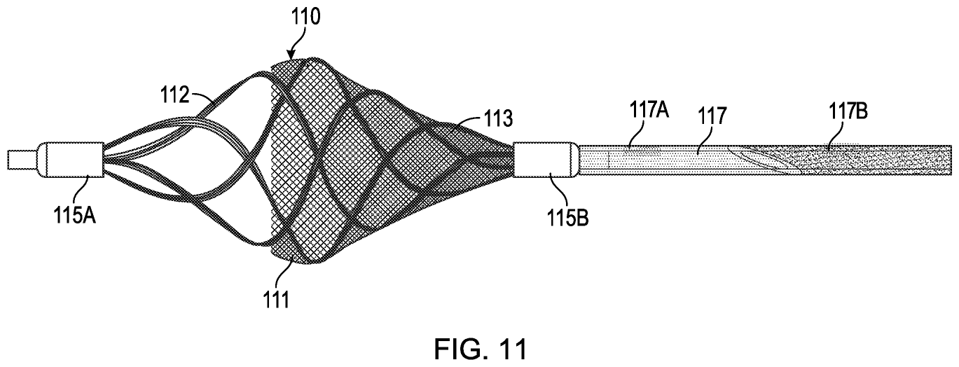

FIG. 11 illustrates a side view of another example embodiment of an embolic protection device 110 comprising a filter 111, a frame 112 to which the filter 111 is attached, a proximal clamp 115A, and a distal clamp 115B. As can be seen, in some embodiments, a tubular member 117 may extends distally from the embolic protection device 110. In the illustrated embodiment, the tubular member 117 is shown as being attached to and extending distally from the distal clamp 115B, though other configurations may be utilized in different embodiments as previously discussed.

Continuing to reference FIG. 11, in some embodiments, it can be seen that the tubular member 117 may comprise an elongated, cylindrical, tubular member. The length of the tubular member 117 may vary. Thus, the relative length of the tubular member 117 in relation to the remaining length of the embolic protection device 110 (e.g., the frame 112) shown in FIG. 11 should not be construed as limiting in scope. In an example embodiment, the overall length of the tubular member 117 may comprise about 10 mm.

As the tubular member 117 forms the leading edge of the embolic protection device 110 during delivery, it may be desirable that at least a portion of the tubular member 117 is flexible or semi-flexible so as to aid with navigation through tortuous anatomy and to gradually transition from a higher stiffness to a lower stiffness. Such a configuration may create a smoother transition in bendability between the distal guidewire tip and the collapsed filter within the delivery catheter housing. Such a configuration may also aid in filling the space between the guidewire 120 and the distal tip of the delivery catheter 130 such as shown in FIG. 2. Thus, as shown in FIG. 11, the elongated tubular member forming the tubular member 117 may comprise a variable durometer or stiffness along its length, with the durometer or stiffness being less along a distal portion of the tubular member 117 than along a proximal portion of the tubular member 117.

In an example embodiment, a first portion 117A of a length of the tubular member 117 may comprise a first durometer or stiffness and a second portion 117B of the length of the tubular member 117 may comprise a second durometer or stiffness. The first portion 117A may comprise a proximal portion and the second portion 117B may comprise a distal portion, with the durometer or stiffness of the first, proximal portion 117A being greater than the durometer or stiffness of the second, distal portion 117B. However, in some embodiments, the reverse configuration may be utilized.

While FIG. 11 illustrates that the first, proximal portion 117A may comprise a first half of a length of the tubular member 117 and that the second, distal portion 117B may comprise a second half of the length of the tubular member 117, it should be appreciated that different ratios may be utilized for the lengths of the respective portions 117A, 117B. In an example embodiment in which the tubular member 117 may comprise a length of about 10 mm, the first, proximal portion 117A may comprise a length of about 5 mm and the second, distal portion 117B may comprise a length of about 5 mm.

The first and second portions 117A, 117B of the tubular member 117 may comprise the same material but with different stiffnesses or durometers, or they may comprise different materials having different stiffnesses or durometers that are fused or attached together using various methods known in the art. By way of example, the first portion 117A of the tubular member 117 may be composed of a thermoplastic elastomer or other polymeric material such as polyether block amide having a first durometer (e.g., PEBAX 53D) and the second portion 117B of the tubular member 117 may be composed of a thermoplastic elastomer or other polymeric material such as polyether block amide having a second durometer (e.g., PEBAX 35D).

In the example embodiment shown in FIG. 11, it can be seen that the first portion 117A and the second portion 117B may each have adjoining, angular (e.g., diagonal or angled) cuts. However, it should be appreciated that, in some example embodiments, the link between the first and second portions 117A, 117B may be linear or vertical (e.g., “butt-bonded”).

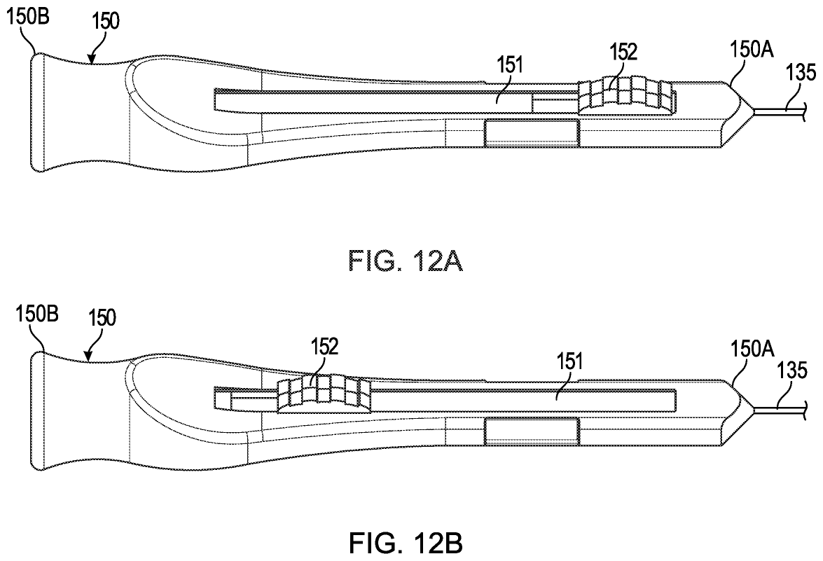

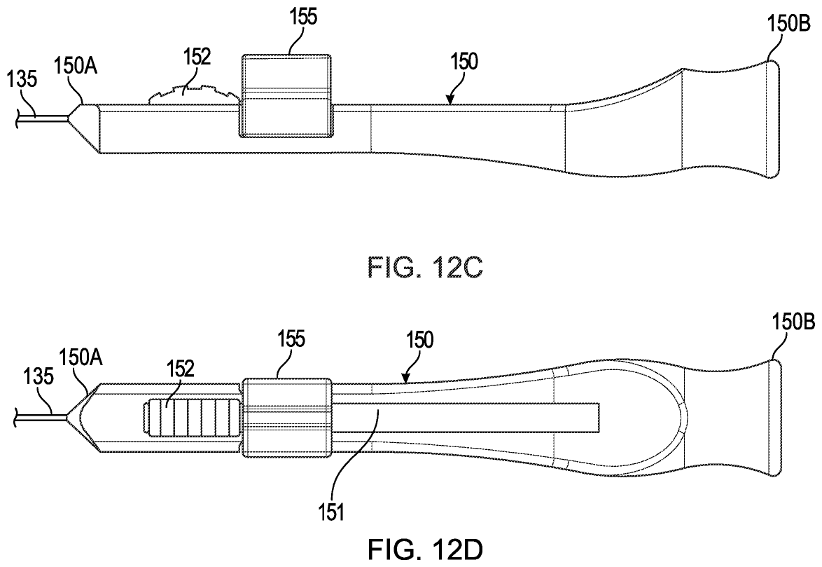

FIGS. 12A, 12B, 12C, and 12D illustrate an example embodiment of a one-handed deployment handle 150 for use with the delivery catheter 130. FIG. 12A illustrates an upper perspective view of a deployment handle 150 of an embolic protection system 100 in an undeployed configuration. FIG. 12B illustrates an upper perspective view of a deployment handle 150 of an embolic protection system 100 in a deployed configuration. FIG. 12C illustrates a side view of a deployment handle 150 of an embolic protection system 100 in an unlocked configuration. FIG. 12D illustrates a top view of a deployment handle 150 of an embolic protection system 100 in a locked configuration.

In the past, such handles for use with delivery catheters have required two-handed operation. By utilizing the example embodiment shown in FIGS. 12A-12D, one-handed operation may be utilized for deployment of the embolic protection device 110 from the delivery catheter 130, thereby freeing the remaining hand of the operator.

As shown in FIGS. 12A-12B, the deployment handle 150 may comprise a substantially elongated configuration having an ergonomic design. In some embodiments, the deployment handle 150 may comprise a proximal end 150B configured to be gripped by a single hand of the operator and a distal end 150A from which the release wire 135 of the delivery catheter 130 may extend. Thus, it should be appreciated that the distal end 150A of the deployment handle 150 may include a lumen through which the release wire 135 may be inserted and secured to a trigger 152 as described in more detail below.

Continuing to reference FIGS. 12A-12B, in some embodiments, the deployment handle 150 may comprise a slot 151 extending along at least a portion of a length of its elongated body. While the figures illustrate that the slot 151 extends along the top of the deployment handle 150, it should be appreciated that other configurations may be utilized (e.g., the slot 151 may instead extend along either side or the bottom of the deployment handle 150).

It should also be appreciated that the length of the slot 151 may vary in different embodiments, and thus the scope should not be construed as being limited to the length of the slot 151 illustrated in the example embodiments shown in the figures. The ratio of the length of the slot 151 with respect to the overall length of the deployment handle 150 may vary in different embodiments.

As further shown in FIGS. 12A-12B, in some embodiments, a trigger 152 may be movably or slidably connected within the slot 151 such that the trigger 152 may be freely movable in either direction (proximally or distally) along at least a portion of a length of the slot 151. The trigger 152 may comprise ergonomic features to aid in griping the trigger 152 with one finger. The release wire 135 of the delivery catheter 130 may be attached or connected (directly or indirectly) to the trigger 152 such that movement of the trigger 152 in the proximal direction pulls the release wire 135 and movement of the trigger 152 in the distal direction pushes the release wire 135.

In some embodiments, the deployment handle 150 may be grasped by a single hand (or by both hands), such as at or near its proximal end 150B, with a one or more fingers or the thumb being utilized to retract the trigger 152 proximally so as to pull on the release wire 135 and thereby expose and expand the embolic protection device 110. FIG. 12A illustrates an upper perspective view the deployment handle 150 and trigger 152 in an undeployed configuration. FIG. 12B illustrates an upper perspective view of the deployment handle 150 and trigger 152 in a deployed configuration.

Turning to FIGS. 12C-12D, in some embodiments, the deployment handle 150 may comprise a locking mechanism 155 operable to lock the trigger 152 in the undeployed configuration and thereby prevent premature deployment of the embolic protection device 110. In some embodiments, the locking mechanism 155 may comprise a tab or other structural component which is hingedly or otherwise adjustable between an unlocked configuration such as shown in FIG. 12C and a locked configuration such as shown in FIG. 12D.

As shown in FIG. 12C, when in the unlocked configuration, the locking mechanism 155 is adjusted out of and away from the path of the trigger 152 along the slot 151. As shown in FIG. 12D, when in the locked configuration, the locking mechanism 155 is adjusted to cross or cover the slot 151 proximally with respect to the trigger 152 when the trigger 152 is in the deployed configuration and thereby prevent the trigger 152 from moving proximally along the slot 151.

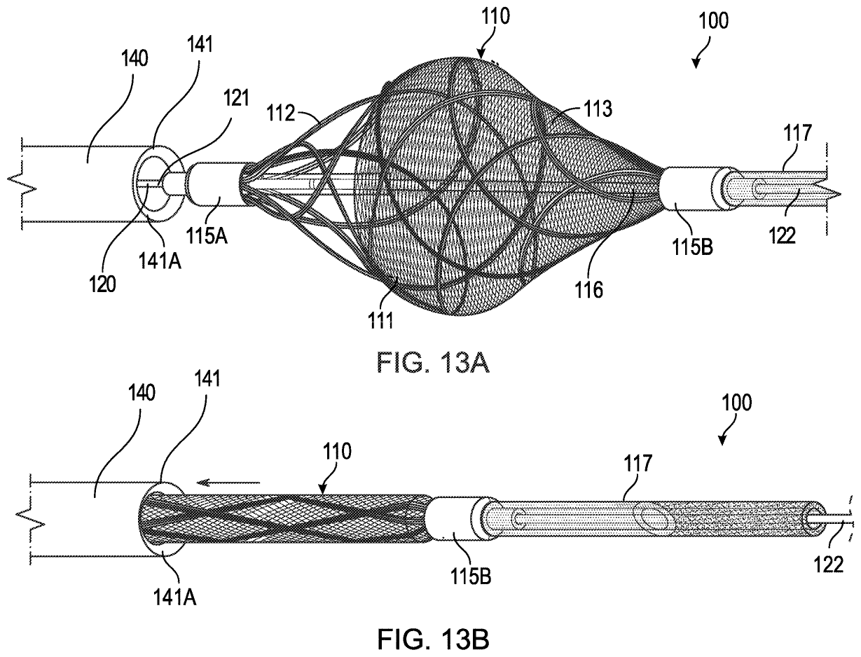

FIG. 13A illustrates a perspective view of a retrieval catheter 140 positioned to retrieve an embolic protection device of an embolic protection system 100. FIG. 13B illustrates a perspective view of an embolic protection device 110 being retrieved by a retrieval catheter 140 of an embolic protection system 100, in accordance with an example embodiment of the present disclosure. As shown in FIGS. 13A-13B, the distal mouth 141 may comprise an inner sloped surface 141A so as to prevent or mitigate fraying of the filter 111 as it enters the distal mouth 141. The inner sloped surface 141A may comprise an inwardly tapered end of the distal mouth 141.

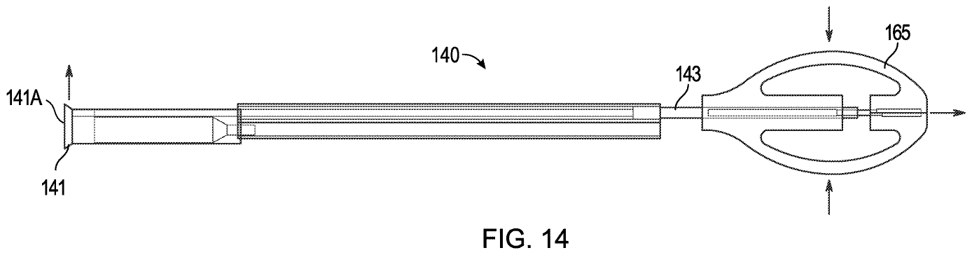

FIG. 14 illustrates a side view of an adjustment handle 165 for use with the retrieval catheter 140 of an embolic protection system 100. In some embodiments, the adjustment handle 165 may be utilized to adjust or deflect a distal tip of the retrieval catheter 140 for improved navigation and to aid in positioning the retrieval catheter 140 to retrieve the embolic protection device 110.

With reference to FIG. 14, in some embodiments, the adjustment handle 165 may be composed of a flexible or semi-flexible material so as to be adjustable between a compressed configuration and an uncompressed configuration. In some embodiments, the adjustment handle 165 may further be composed of a resilient material such that, absent application of force, the adjustment handle 165 naturally reverts to its original shape (e.g., its uncompressed configuration). In some example embodiments, the adjustment handle 165 may function similar to a leaf spring.

In some embodiments, the adjustment handle 165 may comprise a substantially pear-shaped configuration with internal openings such that the adjustment handle 165 may be compressed inwardly. As shown in FIG. 14, in some embodiments, the adjustment handle 165 may also include a lumen extending through its length in which the proximal shaft 143 of the retrieval catheter 140 may be positioned.

The proximal shaft 143 may be anchored within the adjustment handle 165 such that compression of the adjustment handle 165, which causes the adjustment handle 165 to elongate into its compressed configuration, is operable to pull on the proximal shaft 143 and thereby deflect the distal mouth 141 of the retrieval catheter 140 as shown in FIG. 14. Thus, in use, an operator may, with a single hand, compress the adjustment handle 165 to cause deflection of the distal mouth 141 of the retrieval catheter 140 for better navigability in positioning the distal mouth 141 to retrieve the embolic protection device 110.

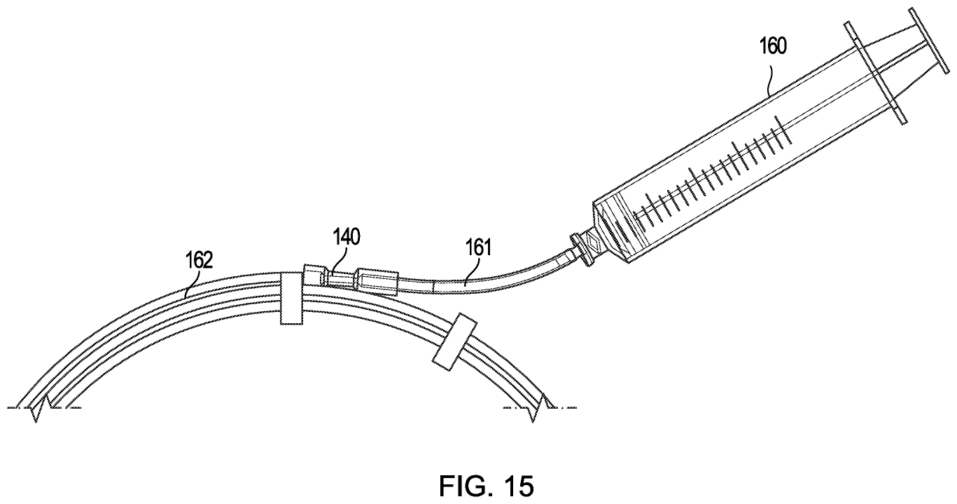

FIG. 15 illustrates a side view of a flushing system for flushing the retrieval catheter 140, such as while the retrieval catheter 140 remains in its manufacturer's packaging. Physicians or other practitioners in the past have been forced to flush out a catheter 140 after it has been removed from its packaging. Often, such a flushing process may be complicated if the physician or other practitioner has shaky hands or is nervous. By flushing the retrieval catheter 140 prior to its removal from its original packaging, the retrieval catheter 140 may be anchored or secured during the flushing process such that any nervousness or shaky hands has no effect on the flushing process.

As shown in FIG. 15, the flushing system may comprise a syringe 160 storing a volume of a fluid. The syringe 160 which may be in fluid communication with tubing 161 that itself may be in fluid communication with the retrieval catheter 140. In some example embodiments, the syringe 160 may be directly connected to the retrieval catheter 140 for flushing without any extra tubing 161. In some embodiments, the tubing 161 may comprise PVC tubing. The retrieval catheter 140 is shown with its distal end being connected to a packaging coil 162 as it may be when in its original packaging.

In use, a fluid (e.g., saline) may be introduced into the syringe 160 and expelled therefrom to flush out the retrieval catheter 140 prior to its removal from its original packaging or removal of the packaging coil 162. The syringe 160 may be connected to a Luer fitting or other port at an end of flexible tubing 161, with the flexible tubing 161 being in fluid communication with the retrieval catheter 140. The plunger of the syringe 160 may then be advanced to expel the fluid through the tubing 161 and the retrieval catheter 140 and thereby flush the retrieval catheter 140. The retrieval catheter 140 may then be removed from its original packaging, ready for use.

In use, the embolic protection device 110 may first be delivered to a target location within a vessel. Generally, the embolic protection device 110 may be delivered to a location which is distal to a location where a medical procedure is to be performed, such as but not limited to a stenting procedure, angioplasty procedure, or any other procedure with a risk of dislodging particles. Example methods of delivery and deployment of an embolic protection device are disclosed in U.S. Pat. No. 11,166,804, which is hereby incorporated by reference in its entirety.

The embolic protection device 110 including the filter 111 may be delivered to the target location by the delivery catheter 130. In some embodiments, the filter 111 may be compressed into its radially compressed configuration and stored entirely within the housing 132 of the delivery catheter 130. The delivery catheter 130 may then be routed to the target location by various methods known in the art.

Upon arrival at the target location, in some embodiments, the release wire 135 of the pull assembly 131 of the delivery catheter 130 may be pulled so as to retract the housing 132 from around the filter 111. As this step is being performed, the push assembly 136 of the delivery catheter 130 may also function to push the filter 111 out of the housing 132. Due to the configuration of the delivery catheter 130, including the use of both pull and push assemblies 131, 136, the filter 111 may be deployed without moving position within a vessel, thereby reducing the filter's 111 tendency to slide out of position while being deployed.

Upon deployment of the filter 111, in some embodiments, the filter 111 may generally expand into its radially expanded configuration distally with respect to the location where the medical procedure is being performed. The delivery catheter 130 may be removed. Any particles which may become dislodged during the medical procedure may enter into the frame 112 of the filter 111 and be caught within the braid 113, thereby preventing various complications caused by such dislodged particles.

Upon completion of the medical procedure, in some embodiments, the filter 111 may be removed from the patient by using the retrieval catheter 140. The retrieval catheter 140 may be advanced up to the embolic protection device 110 and the filter 111 may be pulled through the distal mouth 141 of the retrieval catheter 140 into its housing 142. The wider inner diameter of the distal mouth 141 as compared to the housing 142 may prevent fraying of the filter 111 during its retrieval. The filter 111, upon entering the housing 142, will collapse into its radially compressed configuration. The marker bands 142A, 142B may be utilized to visualize when the filter 111 is fully within the housing 142, and the retrieval catheter 140 may be subsequently removed from the patient's body.

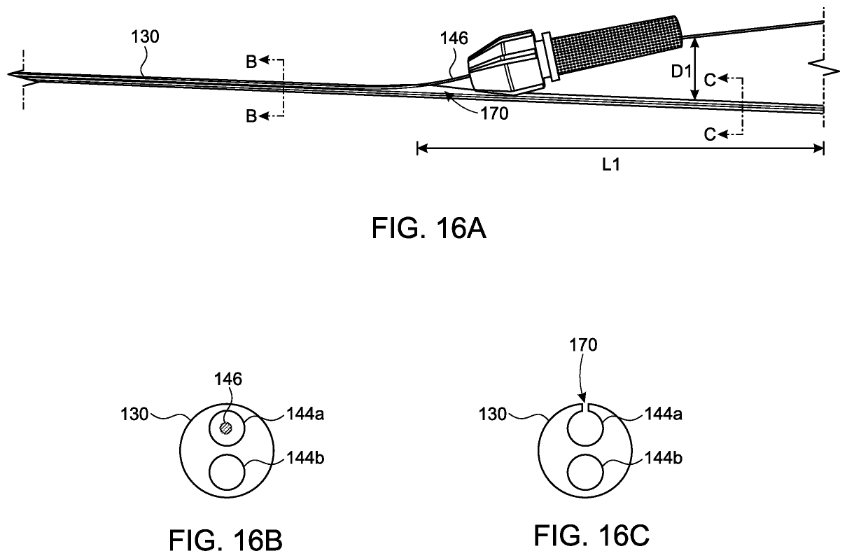

FIGS. 16A-16C illustrate an example of a proximal region of the delivery catheter 130 where a guidewire 146 may be constrained within a lumen (e.g., first lumen 144a or second lumen 144b) of the delivery catheter 130. The delivery catheter 130 may include multiple lumens, for example, a dual-lumen catheter (FIG. 16B) or a tri-lumen catheter (not shown). FIG. 16A illustrates a proximal region of the delivery catheter 130 having multiple lumens. As shown, the guidewire 146 may be contained within a first lumen (e.g., 144a) of the delivery catheter 130, which may leave the second lumen 144b (or third lumen 144c in a tri-lumen catheter, not shown) available as additional working lumen(s). In an example embodiment, the embolic protection device 110 may be navigated to the anatomical target through the second lumen 144b. Using a multi-lumen catheter may allow an operator to better manage the embolic protection device 110, guidewire 146, and any other components that may be inserted into the multi-lumen catheter because the components in the first lumen 144a will be isolated from the components in the second lumen 144b, avoiding entanglement, interference, and/or other adverse interactions.

FIG. 16B illustrates a cross section B-B of the delivery catheter 130 of FIG. 16A. As illustrated, in some embodiments, the delivery catheter 130 may include a first lumen 144a and a second lumen 144b. In some embodiments, the diameter of the first lumen 144a and the second lumen 144b may be the same or different. In one embodiment, the diameter of the first lumen 144a may be larger than the diameter of the second lumen 144b. In some embodiments, the diameter of the first lumen 144a may be less than the diameter of the second lumen 144b. In the illustrated example, the guidewire 146 may be constrained within first lumen 144a. In some embodiments, the second lumen 144b of the delivery catheter 130 may be configured to accommodate the embolic protection device 110 where the embolic protection device 110 can navigate to a target location via the second lumen 144b where a medical procedure is to be performed.

FIG. 16C illustrates a cross section C-C of the delivery catheter 130 of FIG. 16A. As illustrated in FIG. 16C, in some embodiments, the proximal region of the delivery catheter 130 may further include a slit 170 (FIG. 16C) spanning one or more of the multiple lumens. For example, as shown in FIGS. 16A and 16C, the slit 170 may be positioned between the first lumen 144a and the outer surface of the delivery catheter 130 and may run a length L1 from the proximal tip of the delivery catheter 130 to a distal region of the delivery catheter 130. In some embodiments, the slit 170 may begin at the proximal tip and run distally to a location that is proximate to the hemostatic valve. In some embodiments, the slit 170 may extend all the way through the double lumen tubing to an RX port which is distal to the hemostatic valve. As a user pulls out the delivery catheter 130, it may peel away from the guidewire 146.

In some embodiments, the slit 170 may enable the proximal region of the guidewire 146 and guidewire handle to be set aside by a distance D1 away from the delivery catheter which may enhance the ease of operation by providing an operator with enough working space to navigate the embolic protection device 110 to the target location through the second lumen 144b. In one example, the slit 170 runs a length L1 of about 10 cm, 20 cm, 30 cm, 40 cm, or 50 cm.

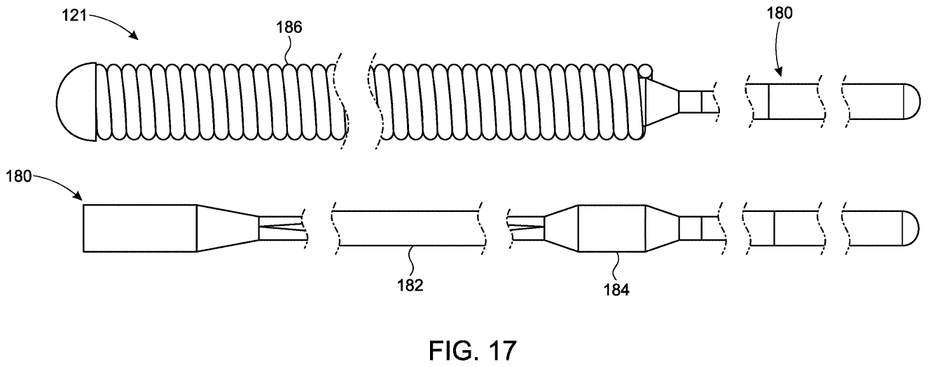

FIG. 17 illustrates an example of a distal end 121 of the guidewire 120 of the embolic protection device 110 as previously discussed above. In some embodiments, the distal end 121 may comprise a core wire 180 with one or more coils 186 affixed over the core wire 180. In the illustrated example, the core wire 180 may comprise one or more reduced diameter regions 182 and/or one or more bulbous regions 184. In some embodiments, the one or more bulbous regions 184 may have a diameter larger than the reduced diameter regions 182 and may taper in the proximate and/or distal direction. In some embodiments, the core wire 180 may be flattened (e.g., rectangular cross-section) which may enhance the bendability of the core wire 180. In some embodiments, the flattened core wire 180 can enhance vascular access by transmitting torque efficiently from proximal end to distal tip of the guidewire 120 and can further facilitate better control and trackability during guidewire tracking. In some embodiments, the one or more coils 186 are affixed to a distal portion of the core wire 180, such as to a distal end thereof, by various methods known in the art such as laser welding. In one example, the one or more coils 186 may be composed of various material including platinum tungsten. In one example, the distal end 121 of the guidewire 120 may comprise a length of about 60-65 mm.

### Clauses

Exemplary embodiments are set out in the following numbered clauses:
Clause 1. An embolic protection system may comprise an embolic protection device including a filter for capturing dislodged particles, a delivery catheter for delivering and deploying the embolic protection device, and a retrieval catheter for retrieving the embolic protection device along with any captured particles.
Clause 2. An embolic protection device may comprise an elongated member such as a guidewire or core wire and a filter movably connected to the elongated member.
Clause 3. An embolic protection device according to clause 2 may comprise a filter which is axially movable with respect to the elongated member.
Clause 4. An embolic protection device according to clauses 2 or 3 may comprise a filter which is rotationally movable with respect to the elongated member.
Clause 5. An embolic protection device according to any of clauses 2-4 may comprise one or more clamps which are movably connected to the elongated member.
Clause 6. An embolic protection device according to clause 5 may comprise a proximal end of the filter being fixed to a first clamp and a distal end of the filter being fixed to a second clamp.
Clause 7. An embolic protection device according to any of the preceding clauses may comprise an anti-clot surface treatment applied to or coated on the filter.
Clause 8. An embolic protection device according to any of the preceding clauses may comprise a filter including a frame formed from one or more structural wires.
Clause 9. An embolic protection device according to clause 8 may comprise a plurality of structural wires, wherein each of the structural wires is comprised of a twinned pair of DFT wires.
Clause 10. An embolic protection device according to clauses 8 and/or 9 may comprise a braid connected to the frame.
Clause 11. An embolic protection device according to any of clauses 8-10 may comprise a braid connected to the frame so as to cover at least half of a length of the frame.
Clause 12. An embolic protection device according to any of the preceding clauses may comprise a filter stopper connected to the elongated member within an interior of the filter.
Clause 13. A delivery catheter may comprise a pull assembly for retracting a housing from around the filter and a push assembly for pushing the filter out of the housing.
Clause 14. A delivery catheter according to clause 13 may comprise a pull assembly including a housing, a marker band, a distal shaft, and/or a release wire.
Clause 15. A delivery catheter according to clauses 13 and/or 14 may comprise a push assembly including a guidewire lumen, a cover tube, and a hypotube.
Clause 16. A method of delivering an embolic protection device may comprise positioning a filter within a housing while the filter is in a radially collapsed or compressed configuration, delivering the housing to a target location, and retracting the housing from around the filter so as to expose and deploy the filter in a radially expanded configuration.
Clause 17. A method according to clause 16 may comprise pushing the filter out of the housing.
Clause 18. A method of delivering an embolic protection device may comprise positioning a filter within a delivery catheter, delivering the delivery catheter to a target vessel, and deploying the embolic protection device from the delivery catheter.
Clause 19. The method according to clause 18 may comprise retracting the delivery catheter from around the embolic protection device using a pull assembly.
Clause 20. The method according to clauses 18 and/or 19 may comprise advancing the embolic protection device out of the delivery catheter using a push assembly.
Clause 21. The method according to any of clauses 18-20 may comprise deploying the embolic protection device at a location that is distal with respect to a location where a medical procedure is being performed.
Clause 22. A method of capturing one or more dislodged particles may comprise delivering and deploying a filter within a vessel so as to capture any of the one or more dislodged particles.
Clause 23. The method according to clause 22 may comprise expanding the filter into a radially expanded configuration and adjusting the radially expanded configuration by axially and/or rotationally moving the filter with respect to an underlying guidewire.
Clause 24. A method of retrieving a filter and any captured particles may comprise delivering a retrieval catheter to a target location, positioning the filter within the retrieval catheter, and retrieving the retrieval catheter from a body of a patient.
Clause 25. The method according to clause 24 may comprise advancing the retrieval catheter over the filter.

Although the disclosure has been described in terms of particular embodiments and applications, one of ordinary skill in the art, in light of this teaching, can generate additional embodiments and modifications without departing from the spirit of or exceeding the scope of the claimed disclosure. Accordingly, it is to be understood that the drawings and descriptions herein are proffered by way of example to facilitate comprehension of the disclosure and should not be construed to limit the scope thereof.

1-42. (canceled)

43. An embolic protection system, comprising:
a filter assembly, the filter assembly comprising:
a guidewire;
a filter movably connected to the guidewire such that the filter may move with respect to the guidewire;
wherein the filter is adjustable between a radially compressed configuration and a radially expanded configuration, wherein the filter comprises a conical shape when in the radially expanded configuration; and
a delivery catheter assembly, the delivery catheter assembly comprising:
a housing for storing the filter assembly prior to deployment; and
a release wire connected to the housing for retracting the housing from around the filter.

44. The embolic protection system of claim 1, wherein the housing includes at least one marker band at or near a distal end thereof.

45. The embolic protection system of claim 1, further comprising a retrieval catheter assembly comprising a retrieval housing for receiving the filter assembly.

46. The embolic protection system of claim 3, wherein the retrieval catheter assembly is comprised of a distal mouth, wherein an inner diameter of the distal mouth is greater than an inner diameter of the retrieval housing.

47. The embolic protection system of claim 3, wherein the retrieval housing comprises a first marker band and a second marker band, the first marker band being positioned at or near a distal end of the retrieval housing and the second marker band being proximally spaced with respect to the first marker band.

48. The embolic protection system of claim 4, wherein the retrieval catheter assembly is further comprised of a proximal shaft extending into a proximal end of the retrieval housing.

49. The embolic protection system of claim 1, wherein the filter is comprised of a plurality of structural wires and a braided mesh.

50. The embolic protection system of claim 1, further comprising a tubular member connected to and extending distally from the filter, the tubular member comprising a proximal end and a distal end, and wherein a durometer of the proximal end of the tubular member is greater than a durometer of the distal end of the tubular member.

51. The embolic protection system of claim 8, wherein the tubular member comprises an elongated cylindrical shape.

52. The embolic protection system of claim 9, wherein a first portion of the tubular member comprises a first durometer, wherein a second portion of the tubular member comprises a second durometer, and wherein the first durometer is greater than the second durometer.

53. The embolic protection system of claim 10, wherein the first portion comprises a first half of a length of the tubular member and wherein the second portion comprises a second half of the length of the tubular member.

54. The embolic protection system of claim 10, wherein the first portion and the second portion are each composed of polyether block amide.

55. The embolic protection system of claim 12, wherein the first portion is composed of PEBAX 53D and wherein the second portion is composed of PEBAX 35D.

56. The embolic protection system of claim 10, wherein the first portion and the second portion are each composed of different materials.

57. The embolic protection system of claim 8, wherein a distal end of the filter is connected to a clamp, and wherein the tubular member is connected to and extends distally from the clamp.

58. An embolic protection system, comprising:
a filter, the filter being adjustable between a collapsed configuration and an expanded configuration;
a delivery catheter assembly, the delivery catheter assembly comprising:
a housing for storing the filter prior to deployment;
a release wire connected to the housing for retracting the housing from around the filter; and
a deployment handle comprising a slot and a trigger movably positioned within the slot, wherein the release wire is connected to the trigger; and,
wherein the trigger is adjustable between an undeployed configuration and a deployed configuration.

59. The embolic protection system of claim 16, wherein the trigger is positioned at or near a distal end of the slot when in the undeployed configuration and wherein the trigger is positioned at or near a proximal end of the slot when in the deployed configuration.

60. The embolic protection system of claim 17, wherein the trigger is operable to pull on the release wire so as to retract the housing when the trigger is in the deployed configuration.

61. The embolic protection system of claim 17, further comprising a locking mechanism, the locking mechanism being adjustable between a locked configuration in which movement of the trigger is minimized or prevented and an unlocked configuration in which the trigger is freely movable along the slot.

62. An embolic protection device, comprising:
an elongated member;
a clamping means movably connected to the elongated member; and
a filtering means for capturing embolic particles;
wherein the filtering means is adjustable between a radially compressed configuration and a radially expanded configuration; and,
wherein the filtering means is connected to the clamping means such that the filtering means may move both radially and rotationally with respect to the elongated member when the filtering means is in the radially expanded configuration.
